(19) United States
(12) Patent Application Publication
(10) Pub. No.: US 2013/0178892 A1
(43) Pub. Date: Jul. 11, 2013

(54) STATIN-LOADED COILS FOR ACCELERATION OF ORGANIZATION AFTER ENDOVASCULAR COILING OF ANEURYSMS

(71) Applicants: Hiroo Iwata, Kyoto-shi (JP); Tomonobu Kodama, Kyoto-shi (JP)

(72) Inventors: Hiroo Iwata, Kyoto-shi (JP); Tomonobu Kodama, Kyoto-shi (JP)

(73) Assignee: Kyoto University, Kyoto (JP)

(21) Appl. No.: 13/823,833

(22) PCT Filed: Sep. 15, 2011
(86) PCT No.: PCT/JP2011/071760

§ 371 Date: Mar. 15, 2013

Related U.S. Application Data
(60) Provisional application No. 61/383,443, filed on Sep. 16, 2010.

(51) Int. Cl. A61L 31/16; A61F 2/01

(52) U.S. Cl. CPC A61L 31/16; A61F 2/01
USPC 606/200

(57) ABSTRACT

The present invention provides a vascular treatment material which has high safety, and also can exert high organization acceleration effect. The present invention also provides a commercially available vascular treatment material, which is easy to produce and can be easily sterilized, and also can be stored for a long period. Disclosed is a vascular treatment material including a coil on which statin is loaded. It is preferred that a wire forming the coil is made of at least one kind of metal selected from platinum, tungsten, gold, cobalt, chromium, titanium, niobium, aluminum, tantalum, iron, nickel and the like. The vascular treatment material of the present invention is particularly useful, which is placed in a blood vessel for embolization treatment, or which accelerates organization for prevention of recanalization of aneurysm or for prevention of rupture of aneurysm.

### CROSS-REFERENCE TO RELATED APPLICATIONS

This application claims benefit under Paris Convention or 35 U.S.C. §119(e) of U.S. Provisional Application No. 61/383,443 filed Sep. 16, 2010, incorporated herein by reference in its entirety.

### TECHNICAL FIELD

The present invention relates to a vascular treatment material including a coil on which statin is loaded. The vascular treatment material according to the present invention is easy to produce and particularly exerts excellent endovascular organization effect, and therefore can be preferably utilized as a material for embolization treatment.

### BACKGROUND ART

In vascular disorders such as aneurysm, arteriovenous malformation and arteriovenous fistula, and in an embolization treatment (or therapy) of the nutrient artery to tumor or the like, an endovascular treatment is performed using an embolization material. That is, a tip of a catheter is directed to the vicinity of the treatment site and the embolization material is injected into the treatment site through the tip. Alternatively, a guide wire containing the embolization material in the tip is inserted into the treatment site through this catheter, and thus the embolization material portion is separated and is retained at the treatment site. Blood flow to the treatment site is blocked by the embolization material per se and thrombus formed on the embolization material, and the treatment is performed. A fine coil made of a platinum alloy is suitably used as such the embolization material.

Efficacy and safety of the treatment method are apparent. Recently, cases where a coil is applied as the embolization material have quickly increased and the limitation has gradually become apparent. For example, in the treatment of aneurysm, thrombus is formed in the aneurysm by controlling a volume fraction of the coil retained in the aneurysm to 30% or more so as to prevent blood from flowing into the aneurysm. Then, fibroblasts, smooth muscle cells and the like proliferate, the thrombus is organized in the aneurysm and the entrance of the aneurysm is also covered with vascular endothelial cells, and thus it was expected that healing arises. However, when treatment sites of an animal experimental model and an example in pathologic autopsy are observed, the circumference of the coil is not organized even after a lapse of a long period after retaining the coil. In some cases, recanalization of the aneurysm arises and the treatment effect does not increase, and thus blood flowing into the aneurysm may sometimes cause rupture of the aneurysm. In the orifice of the aneurysm, the coil is often in contact directly with the blood flow. In such a state, the thrombus is formed on the coil of the orifice and this thrombus is exfoliated and the blood vessel is occluded, and cerebral infarction may be caused. Therefore, it was desired to develop a coil which accelerates organization of thrombus on the circumference of the coil and accelerates endothelialization of the orifice of the aneurysm.

Non-Patent Documents 1 and 2 disclose, as the coil which accelerates organization, a platinum coil subjected to ion implantation, and a coil in which collagen has been adsorbed on the platinum coil subjected to ion implantation, and reports that these coils are effective in an animal experiment. However, these coils merely have improved adhesion of cells on surfaces of the coils, and do not positively accelerate organization. Since the ion implantation method with high costs is used, these coils are expensive and not spread.

In contrast, Patent Document 1 discloses a coil in which a cell growth factor or a vector containing a gene of the cell growth factor is fixed on a surface of the coil. This is considered as follows. The cell growth factor is gradually released from the coil by fixing the cell growth factor on the surface of the coil, or circumferential cells are infected with the vector containing the gene of the cell growth factor by fixing the vector on the surface of the coil and then the cell growth factor is gradually released from the cells, and thus proliferation of the cells are accelerated at the treatment site, resulting in acceleration of organization. It is expected that use of these coils enables to carry out embolization treatment more surely, and also to accelerate the organization and prevent the rupture of the aneurysm.

However, it is necessary to fix the cell growth factor or the vector containing the gene thereof on the surface of the coil so as not to be flowed away by the blood flow in order that the coil disclosed in Patent Document 1 effectively functions. For that purpose, there is required a troublesome treatment of binding the cell growth factor or the vector containing the gene thereof by an interionic interaction after forming a film having a polar group on the surface of the coil. There is a problem that, most of the cell growth factor or the vector containing the gene thereof is deactivated when the other fixation method, for example, chemical bonding is performed. There is also a problem that, even if the cell growth factor or the vector containing the gene thereof can be chemically bound, the cell growth factor or the vector is unstable and is likely to be deactivated and therefore it is difficult to be sterilized or is inferior in storability.
Patent Document 1: JP 2001-299769 A
Non-Patent Document 1: Y. Murayama, Y. Suzuki, F. Vinuela et al., Development of a Biologically Active Guglielmi Detachable Coil for the Treatment of Cerebral Aneurysms. Part I: In Vitro Study, AJNR Am J. Neuroradiol, 20, 1986-1991 (1999)
Non-Patent Document 2: Y. Murayama, F. Vinuela, Y. Suzuki, Development of a Biologically Active Guglielmi Detachable Coil for the Treatment of Cerebral Aneurysms. Part II: An Experimental Study in a Swine Aneurysm Model, AJNR Am J. Neuroradiol, 20, 1992-1999 (1999)

### DISCLOSURE OF THE INVENTION

### Problems to be Solved by the Invention

An object of the present invention is to provide a vascular treatment material (or a material for curing blood vessel) which has high safety, and also can exert high organization acceleration effect. Another object of the present invention is to provide a commercially available vascular treatment material which is easily produced and can be easily sterilized, and also can be stored for a long period.

### Means for Solving the Problems

The present inventors has intensively studied and found, surprisingly, that a coil is coated with a solution of statin, which statin has a long history of being administered to human and has secured safety thereof, and then the coil is dried to simply produce a statin-loaded coil having high safety, and that embolization treatments can be performed by placing the statin-loaded coil in a blood vessel using the same method as that of the prior art, and surprisingly, the statin-loaded coil can remarkably accelerate organization to prevent recanalization of aneurysms and to prevent rupture of aneurysms. Thus, the present invention has been completed.

In an aspect of the present invention, a vascular treatment material (or a material for curing blood vessel) including a coil on which statin is loaded (or at least a part is coated with statin) is provided.

In an embodiment of the present invention, the treatment material in which a wire forming the coil is made of at least one kind of metal selected from platinum, tungsten, gold, cobalt, chromium, titanium, niobium, aluminum, tantalum, iron and nickel is provided.

In another embodiment of the present invention, the treatment material is provided, wherein the wire forming the coil has a diameter of 120 to 120 μm and the coil has a diameter of 100 to 500 μm.

In still another embodiment of the present invention, the treatment material in which the coil has a length in a longitudinal direction of 10 to 300 mm is provided.

In preferred embodiment of the present invention, the treatment material is provided, wherein statin includes at least one kind selected from simvastatin, pravastatin, fluvastatin, lovastatin, mevastatin, cerivastatin, atorvastatin, pitavastatin and rosuvastatin.

In still another embodiment of the present invention, the treatment material is provided, wherein the treatment material is placed (or retained) in a blood vessel for embolization treatment, or the treatment material accelerates organization for prevention of recanalization of aneurysms or for prevention of rupture of aneurysms.

In another aspect of the present invention, a method for producing the vascular treatment material is provided, wherein the method includes a step of immersing a coil in a statin-containing solution and then drying the coil, or a step of coating a coil with a statin solution and then drying the coil.

### Effects of the Invention

A vascular treatment material according to the present invention includes a coil on which statin is loaded, and therefore has high safety and can exert high organization acceleration effect. The vascular treatment material is particularly excellent as an embolization treatment material. Furthermore, it is easy to produce the vascular treatment material since it is possible to produce by immersing a coil in a statin-containing solution or coating a coil with a statin-containing solution.

### BRIEF DESCRIPTION OF THE DRAWINGS

FIG. 1 is a schematic view for explaining a state where a vascular treatment material of the present invention is placed in an aneurysm of a blood vessel.

FIG. 2 shows electron micrographs of vascular treatment materials and insertion thereof into carotid arteries.

FIG. 2(a-1) shows an electron micrograph of a vascular treatment material of Comparative Example 1, and FIG. 2(a-2) shows an electron micrograph of a vascular treatment material of Example 1. Each of the vascular treatment materials has a diameter of about 300 μm.

FIG. 2(b-1) schematically shows a carotid bifurcation of Wister rats. First, a common carotid artery branches into an internal carotid artery (left) and an external carotid artery (right). An ascending pharyngeal artery branches to the left side from the external carotid artery, and also a superior thyroid artery branches to the right side.

FIG. 2(b-2) shows a visual photograph of an incised carotid bifurcation. FIG. 2(b-3) shows the carotid bifurcation after insertion of a vascular treatment material. An arrow indicates an external carotid artery into which a vascular treatment material was inserted. A scale bar was 4 mm.

FIG. 3 shows photographs of bifurcations (aneurysm models) of carotid arteries dissected on the 14th day after placing vascular treatment materials. FIG. 3(a-1) shows a bifurcation in which a vascular treatment material of Comparative Example 1 was placed, FIG. 3(a-2) shows a bifurcation in which a vascular treatment material of Example 1 was placed, FIG. 3(b-1) shows a photograph of an orifice of an external carotid artery in which the vascular treatment material of Comparative Example 1 was placed, and FIG. 3(b-2) shows a photograph of an orifice of an external carotid artery in which the vascular treatment material of Example 1 was placed.

FIG. 4 shows images stained with eosin hematoxylin (HE) and Masson trichrome (MT) of the cross sections of the incised portions on the 14th day after placing the vascular treatment materials. FIG. 4(a) shows a pathologic photograph when the treatment material of Comparative Example 1 was implanted. FIG. 4(b) shows a pathologic photograph when the treatment material of Example 1 was implanted. When FIGS. 4(a) and 4(b) are compared, in the treatment material of Example 1, the cell component apparently occupies so as to occlude inside the aneurysm. As is apparent from pathologic evaluation, the inside was completely filled with smooth muscle cells and extracellular matrix.

FIG. 5 shows a case where, with respect to the aneurysm model in which the vascular treatment material of Example 1 was implanted, a cross section of the incised portion on the 14th day after placing the vascular treatment material was subjected to immunostaining regarding a Smooth Muscle Actin (αSMA) and von Willebrand Factor (vWF). It is apparent that vWF was stained and the orifice of the aneurysm was covered with endothelial cells (upper column), while the photograph (lower column) shows that αSMA was stained and smooth muscle cells were present inside the aneurysm.

FIG. 6 shows cases where a segment inside the aneurysm was made at axial ligation. FIG. 6(a) shows the case where the vascular treatment material of Comparative Example 1 was used, and FIG. 6(b) shows the case where the vascular treatment material of Example 1 was used. FIG. 6(1) shows the case where 2 weeks have passed after implantation. FIG. 6(2) shows the case where 4 weeks have passed. In both drawings, remarkable organization inside the aneurysm was recognized in the case where vascular treatment material of Example 1 was implanted.

FIG. 7 shows organization rates inside the aneurysms which are expressed by %. A white bar shows the case of the vascular treatment material of Comparative Example 1, and a black bar shows the case of the vascular treatment material of Example 1. It is apparent that organization was significantly accelerated when the vascular treatment material of Example 1 was used in both cases of the 2nd week and 4th week after implantation.

FIG. 8 shows organization rates inside the aneurysms in case of using the vascular treatment materials of Examples 2 to 7 which are expressed by % from the left side. Each drawing shows the case of the 2nd week after implantation. When compared with Comparative Example 1 shown in FIG. 7, it is apparent that organization was significantly accelerated when the vascular treatment materials of Example 2 to 7 were used.

FIG. 9 are photographs each of which shows a cross section obtained by cutting the aneurysm together with the coil, at the position of 2 mm from the orifice of the aneurysm on the 2nd week after implantation when the vascular treatment material of Examples 2 to 7 were used. The photographs (upper left to lower left) show Examples 2 to 4, and the photographs (upper right to lower right) show Examples 5 to 7. When compared with Comparative Example 1 shown in FIG. 3(b-1), it is apparent that the circumference of each coil was surrounded by a white tissue and organization was significantly accelerated in each case.

### MODE FOR CARRYING OUT THE INVENTION

The vascular treatment material according to the present invention includes statin and a coil, and statin is loaded on the coil.

In the present invention, “statin” means a drug which decreases cholesterol level in blood by inhibiting a function of HMG-CoA reductase. There is no particular limitation on statin as long as the objective vascular treatment material of the present invention can be obtained.

In “statin” according to the present invention, pharmaceutical acceptable compounds of statins are included. Specific examples of the statins include simvastatin (or (1S,3R,7S,8S,8aR)-8-{2-[(2R,4R)-4-hydroxy-6-oxotetrahydro-2H-pyran-2-yl]ethyl}-3,7-dimethyl-1,2,3,7,8,8a-hexahydronaphthalen-1-yl2,2-dimethylbutanoate: commercially available from BANYU PHARMACEUTICAL CO., LTD/Merck & Co., Inc.), pravastatin (or (3R,5R)-3,5-dihydroxy-7-((1R,2S,6S,8R,8aR)-6-hydroxy-2-methyl-8-{[(2S)-2-methylbutanoyl]oxy}-1,2,6,7,8,8a-hexahydronaphthalen-1-yl)-heptanoic acid: commercially available from Daiichi Sankyo Healthcare Co., Ltd./Bristol-Myers Squibb), lovastatin (or (1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl(2S)-2-methylbutanoate: commercially available from Shionogi & Co., Ltd./AstraZeneca), fluvastatin (or (3R,5S,6E)-7-[3-(4-fluorophenyl)-1-(propan-2-yl)-1H-indol-2-yl]-3,5-dihydroxyhept-6-enoic acid: commercially available from Novartis Pharma/Mitsubishi Tanabe Pharma Corporation), mevastatin (or (1S,7R,8S,8aR)-8-{2-[(2R,4R)-4-hydroxy-6-oxotetrahydro-2H-pyran-2-yl]ethyl}-7-methyl-1,2,3,7,8,8a-hexahydronaphthalen-1-yl(2S)-2-methylbutanoate), cerivastatin (or (3R,5S,6E)-7-[4-(4-fluorophenyl)-5-(methoxymethyl)-2,6-bis(propan-2-yl)pyridin-3-yl]-3,5-dihydroxyhept-6-enoic acid), atorvastatin (or (3R,5R)-7-[2-(4-fluorophenyl)-3-phenyl-4-(phenylcarbamoyl)-5-propan-2-ylpyrrol-1-yl]-3,5-dihydroxyheptanoic acid: commercially available from Astellas Pharma Inc./Pfizer Inc.), pitavastatin (or (3R,5S,6E)-7-[2-cyclopropyl-4-(4-fluorophenyl)quinolin-3-yl]-3,5-dihydroxyhept-6-enoic acid: commercially available from KOWA PHARMACEUTICAL COMPANY LTD.) and rosuvastatin (or (3R,5S,6E)-7-[4-(4-fluorophenyl)-2-(N-methylmethanesulfonamido)-6-(propan-2-yl)pyrimidin-5-yl]-3,5-dihydroxyhept-6-enoic acid: commercially available from Merck & Co., Inc.).

It is more preferred that statin includes simvastatin, pravastatin, lovastatin, fluvastatin, atorvastatin, pitavastatin and rosuvastatin.

It is possible to use, as these statins, commercially available statins, and these statins can be used alone or in combination.

In the present invention, “coil” means a substance obtained by spirally winding a wire which, may have various cross sections, and there is no particular limitation on the coil as long as the statin can be loaded thereon and the objective vascular treatment material of the present invention can be obtained.

Size and shape of the coil may be the same as those of coils of embolization materials which have hitherto been used in embolization treatment (or therapy).

Diameter of a cross section in a direction vertical to a major axis of the coil (hereinafter may also be referred to as a “diameter of the coil”) and length in a longitudinal direction can be appropriately adjusted corresponding to size of a site of a blood vessel to be used.

The diameter of the coil is generally, for example, from 100 to 500 μm and the length in the longitudinal direction of the coil is generally, for example, from 10 to 300 mm.

Such the coil can be adequately produced from a wire material. Specifically, diameter of the wire material is preferably from 20 to 120 μm, and more preferably from 30 to 60 μm. The “coil” may be produced, for example, by directly winding this wire material. It is also possible to obtain the “coil” according to the present invention by once making a “primary coil” having a diameter of 100 to 2,000 μm, and then rewinding so as to become the diameter which fits the inner diameter of the treatment site of the blood vessel to which the coil is to be applied. In some cases, the “coil” may be produced after making a “secondary coil” having a diameter of 2 to 30 mm from the “primary coil”.

There is no particular limitation on the wire material as long as it can be visually confirmed from the outside of the body by some method and has biocompatibility to some extent, and also has mechanical characteristics capable of forming the coil and is capable of obtaining the vascular treatment material according to the present invention.

Examples of such a wire material include metal, a polymer material and the like. The wire material is preferably metal which can be visually confirmed, satisfactorily, under radioscopy utilized during surgery.

Examples of “metal” include at least one kind of metal selected from platinum, tungsten, gold, cobalt, chromium, titanium, niobium, aluminum, tantalum, iron and nickel, and alloys made of a combination of these metals, which are preferred. The “metal” is more preferably at least one kind selected from the group consisting of platinum, stainless steel, tantalum, a tantalum alloy, a platinum-tungsten alloy, a platinum-gold alloy, gold, a gold alloy and a cobalt-based chromium alloy. Among these metals, a platinum-tungsten alloy, stainless steel and a platinum-gold alloy are particularly preferred and a platinum-tungsten alloy and a platinum-gold alloy are most preferred since they have less in vivo reactivity and scarcely exhibit toxicity, and also have mechanical properties suited for the endovascular operation.

At least a part of the coil according to the present invention may be coated or not coated with other materials, for example, a biocompatible material, a biodegradable material, a polymer material such as a synthetic resin, a ceramics material and the like. There is no particular limitation on other materials as long as the objective vascular treatment material of the present invention can be obtained. Particularly, materials, which have hitherto been used clinically as in vivo implant materials, are more preferred.

Examples of the other material include an ethylene-vinyl acetate copolymer, polyester, a silicone rubber (RTV rubber), a thermoplastic polyurethane, a fluororesin (for example, PTFE, ETFE, thermoplastic fluororesin, etc.), polyolefin (for example, polyethylene, polypropylene, low density polyethylene, low density polypropylene, etc.), polyester, polycaprolactone, polyvinyl acetate, polycarbonate, polyimide carbonate, aliphatic polycarbonate, silicone, a mixture of polyether type polyurethane and dimethyl silicone and a block copolymer, polyurethane such as segmented polyurethane, polyacrylamide, polyethylene oxide, polycarbonate such as polyethylene carbonate and polypropylene carbonate, polymethyl methacrylate, polybutyl methacrylate, polymethoxyethyl acrylate, polyhydroxyethyl methacrylate, a copolymer of hydroxyethyl methacrylate and styrene (for example, HEMA-St-HEMA block copolymer), a mixture thereof and the like.

It is possible to use, as the polymer material, a biodegradable material which is in vivo enzymatically or non-enzymatically degraded in which the degraded product does not exhibit toxicity. Examples of the biodegradable material include polylactic acid, polyglycolic acid, polycaprolactone, polyhydroxybutyric acid, poly α-amino acid, polymalic acid, and a copolymer thereof (for example, a polylactic acid-polyglycolic acid copolymer, a polylactic acid-polycaprolactone copolymer and the like), saccharides, fibrin, collagen, gelatin, laminin, heparansulfuric acid, fibronectin, vitronectin, chondroitinsulfuric acid, hyaluronic acid, chitin, chitosan, a mixture thereof, and the like.

In the vascular treatment material of the present invention, the portion of the coil on which statin is loaded may be coated with a polymer material or not. Furthermore, the portion of the coil on which statin is loaded may be coated with a mixture of statin and the polymer material or not. There is no particular limitation on the method of loading statin on the coil as long as the objective vascular treatment material of the present invention can be obtained. Examples thereof include a method in which a solution containing statin dissolved therein is prepared and a surface of a coil is coated with the thus prepared solution and then the coil is dried, and a method in which a coil is immersed in the solution and then dried and the like.

The solution containing statin dissolved therein can be prepared by appropriately mixing a solvent with statin. There is no particular limitation on the solvent as long as it does not react with statin and does not make statin unstable, and also stably dissolve statin and has a moderate boiling point which enables easy vaporization later, and is also capable of obtaining the objective vascular treatment material of the present invention. Examples of the solvent include alcohols such as methanol, ethanol and propanol; ketones such as acetone; ethers such as dioxane and tetrahydrofuran; nitriles such as acetonitrile; and the like.

There is no particular limitation on the concentration of statin in the statin solution as long as the objective vascular treatment material of the present invention can be obtained. Taking the amount of statin loaded on the coil into consideration, the concentration is preferably comparatively high concentration and is preferably from 5 to 45% by weight, and more preferably from 15 to 35% by weight.

There is no particular limitation on the amount of statin loaded on the coil surface as long as the objective vascular treatment material of the present invention can be obtained, and the amount of statin is preferably from 0.1 μg to 500 μg/10 mm·coil, and more preferably from 5 to 60 μg/10 mm·coil. When the amount of statin is less than 0.1 μg/10 mm·coil, the organization acceleration effect can become insufficient. When the amount of statin is more than 500 μg/10 mm·coil, it can become uneconomical. “10 mm·coil” means per 10 mm in length in a longitudinal direction of the coil.

The amount of statin loaded can be determined from an increase in weight by weighing the weight of the coil before and after loading statin on the coil.

In the present description, “load” means a state where statin is positioned (or placed) on the coil, and means that statin adheres to the coil to cover at least a part of the coil.

There is no particular limitation on the loading method and the state after loading, and loading may be physically or chemically performed as long as the objective vascular treatment material of the present invention can be obtained.

It is possible to accelerate organization of the circumference of the vascular treatment material by placing the vascular treatment material of the present invention in a blood vessel, particularly a blood vessel (or vascular) disorder site. Herein, “blood vessel disorder site” means a site having some disorder generated in the blood vessel, and there is no particular limitation on the site as long as the vascular treatment material of the present invention can be utilized. Examples thereof include aneurysm, arteriovenous malformation, arteriovenous fistula and the like. The vascular treatment material of the present invention is also useful for embolization treatment of nutrient artery communicated with tumor.

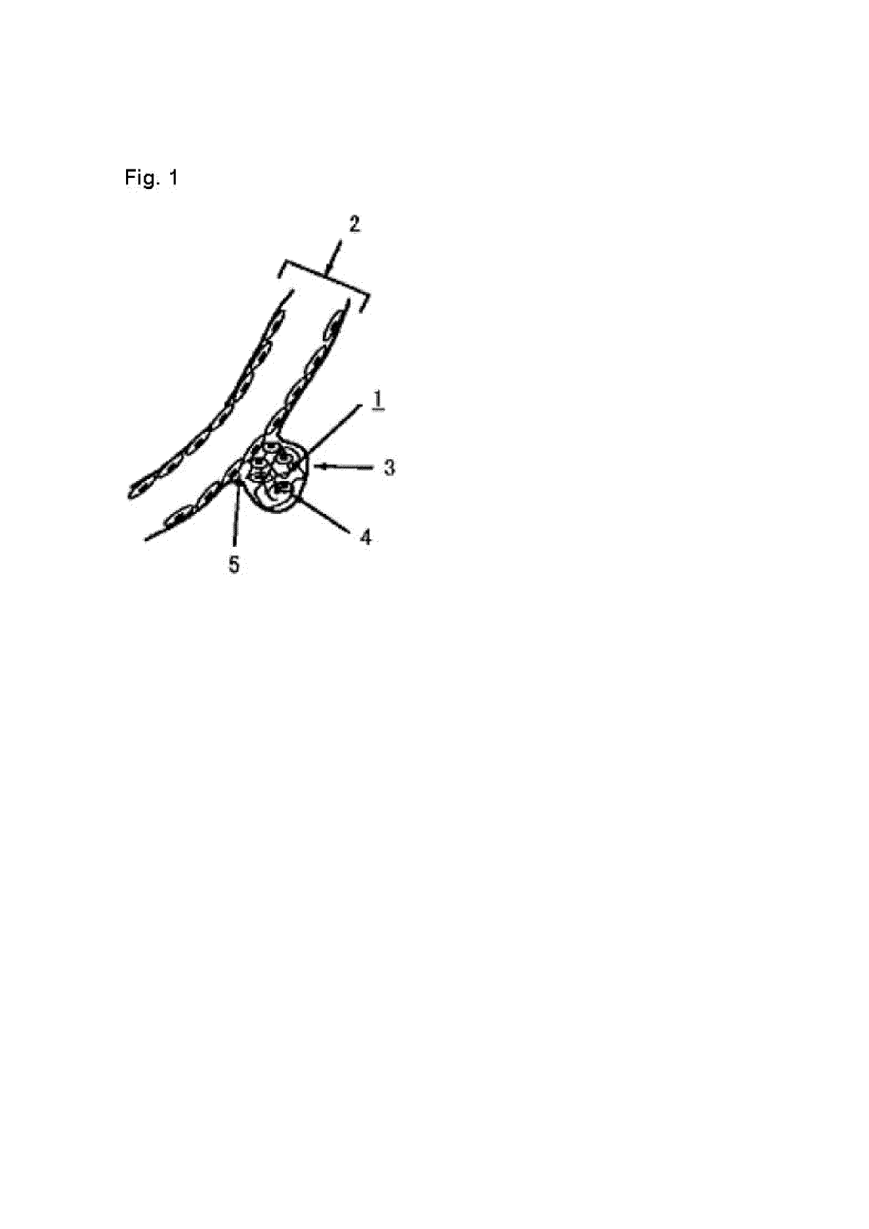

The endovascular function of the vascular treatment material of the present invention will be described with reference to FIG. 1.

FIG. 1 is a schematic view for explaining a state where a vascular treatment material of the present invention is placed in an aneurysm of a blood vessel. In a blood vessel 2, an aneurysm 3 is generated. A vascular treatment material 1 is placed inside the aneurysm 3. By placing the vascular treatment material 1, fibroblasts or smooth muscle cells 4 proliferate in the aneurysm 3 and inside the aneurysm 3 is organized. Furthermore, the orifice of the aneurysm 3 is covered with new vascular endothelial cells 5. As a result, the aneurysm 3 is substantially blocked from the blood vessel 2, and thus scattering of thrombus formed in the aneurysm 3 and rupture of the aneurysm 3 can be effectively prevented.

The reason why the present invention exerts excellent effect is considered as follow, but the present invention is not limited by the following reason.

Statin such as simvastatin exhibits the hypolipidemic activity and is used for healing hyperlipemia. Furthermore, it is known that statin such as simvastatin has not only hypolipidemic activity but also wound healing accelerating activity for healing wounds such as surgical dissection, gastric ulcer, burn and laceration (please refer to Non-Patent Document: J Biol Chem. 2008 May 30; 283(22): 15479-90. Epub 2008 Apr. 3. Biphasic regulation of HMG-CoA reductase expression and activity during wound healing and its functional role in the control of keratinocyte angiogenic and proliferative responses. Schiefelbein D, Goren I, Fisslthaler B, Schmidt H, Geisslinger G, Pfeilschifter J, Frank S.), and induces production of a transforming growth factor (TGF-β) as a kind of cytokines (please refer to Non-Patent Document: Nyan M, Miyahara T, Noritake K, Hao J, Rodriguez R, Kuroda S, Kasugai S.: Molecular and tissue responses in the healing of rat calvarial defects after local application of simvastatin combined with alpha tricalcium phosphate. J Biomed Mater Res B Appl Biomater. 2010 April; 93(1): 65-73).

In contrast, statin such as simvastatin is remarkably stable when compared with a cell growth factor or a vector containing a gene thereof. Furthermore, statin is easily dissolved without being deactivated by organic solvents such as ethanol, methanol and acetonitrile. Therefore, statin loaded on the cell is remarkably stable and is therefore excellent in handling, usability and the like.

Accordingly, since statin such as simvastatin can be handled stably and easily until statin is placed in the blood vessel, it can be effectively arranged inside the blood vessel. It is considered that statin can induce TGF-β1 or the like from cells by direct or indirect action of the drug on the cells of platelets, macrophages and the like. As a result, it is considered that the induced TGF-β1 accelerates organization, and thus exerting the above-mentioned effect.

### EXAMPLES

Hereinafter, the present invention will be described in more detail and specifically by way of Examples and Comparative Examples, which are for illustrative purpose only, and the present invention is not limited to these Examples.

### Example 1

Simvastatin was dissolved in ethanol to prepare a 26.5% wt % ethanol solution. Meanwhile, a platinum-tungsten (8%) alloy wire having a wire diameter of 45 μm was wound to obtain a platinum coil having a diameter of 300 μm and a length of 10 mm. The obtained platinum coil was immersed in the ethanol solution of simvastatin (commercially available from BANYU PHARMACEUTICAL CO., LTD.) and then dried to obtain a vascular treatment material of Example 1. Using a precision balance, the following measurement was performed. The amount of simvastatin loaded on the platinum coil was about 50 μg/10 mm·coil.

### Comparative Example 1

In the same manner as in Example 1, a platinum-tungsten (8%) alloy wire having a wire diameter 45 μm was wound to obtain a platinum coil having a diameter of 300 μm and a length of 10 mm. The obtained platinum coil was used as a vascular treatment material of Comparative Example 1 as it is without loading simvastatin.

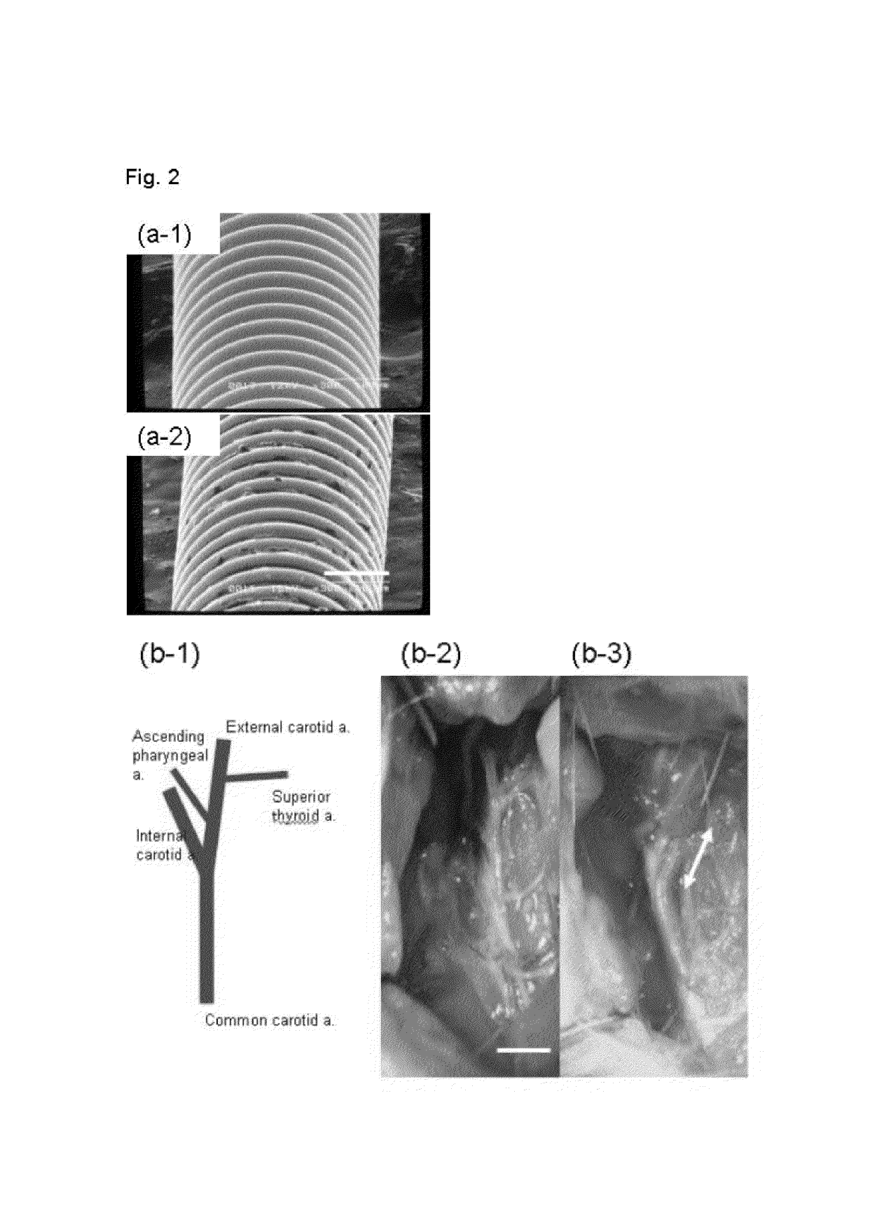

In FIG. 2, electron micrographs (SEM) of the vascular treatment materials of Example 1 and Comparative Example 1 are shown. FIG. 2(a-1) shows the vascular treatment material of Comparative Example 1, and FIG. 2(a-2) shows the vascular treatment material of Example 1. In FIG. 2(a-2), a state where a crystal of simvastatin adheres to the coil was recognized.

### Evaluation Method

External carotid arteries of Wister rats (male, weighing 300 to 350 g) were ligated at 5 mm from carotid bifurcations and these blind-ended external carotid arteries were used as a model of aneurysm. The vascular treatment materials produced in Example 1 and Comparative Example 1 were placed (indwelled or retained) in this aneurysm model. With respect to the respective vascular treatment materials, six Wister rats were respectively used. On the 14th day and 28th day after placing the vascular treatment materials, the rats were sacrificed and corresponding blood vessels were taken out (or removed).

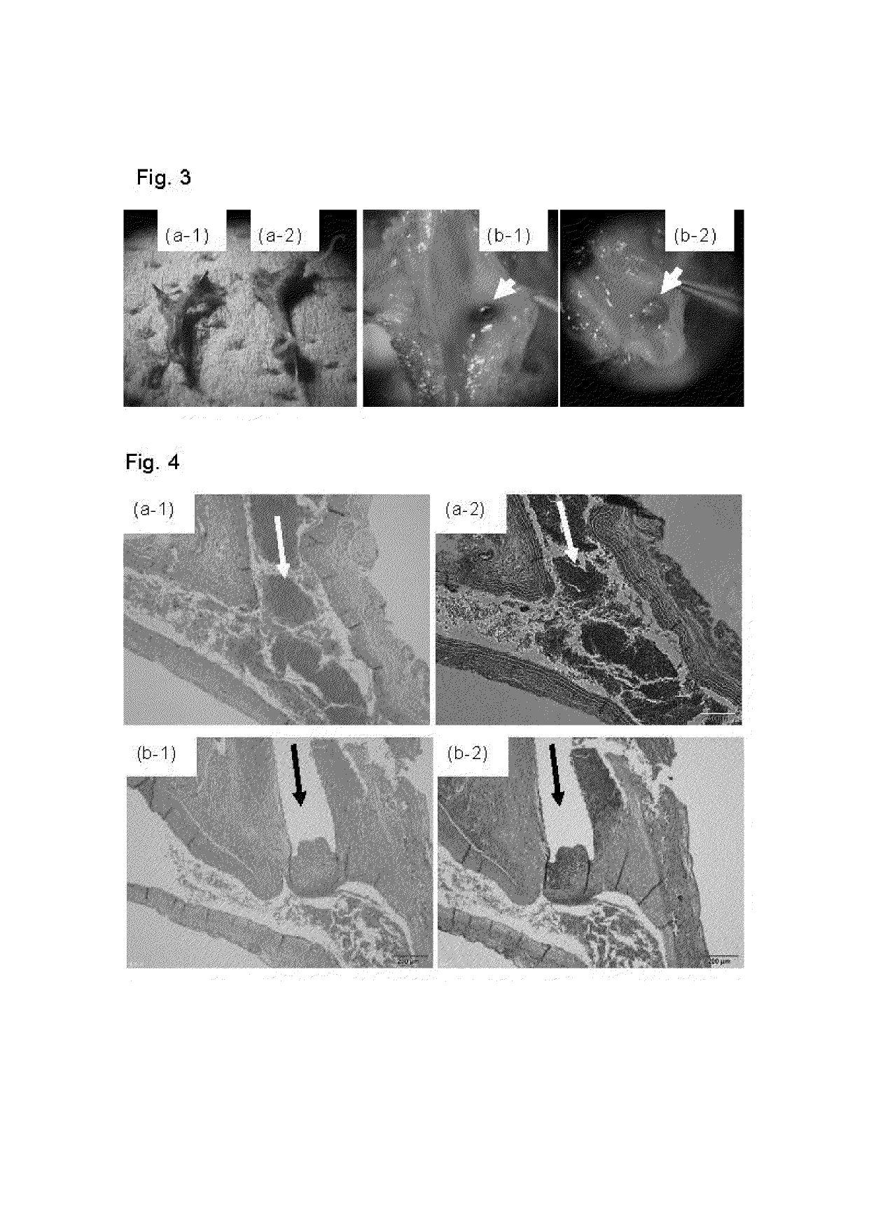

FIG. 2(b-1) schematically shows the carotid bifurcation of the Wister rat. The common carotid artery branches into the internal carotid artery (left) and the external carotid artery (right). This branched external carotid artery was used as the aneurysm model. FIG. 2(b-2) shows a photograph of the dissected carotid bifurcation. FIG. 2(b-3) shows the carotid bifurcation after inserting a vascular treatment material. The vascular treatment material was inserted into the external carotid artery.

In FIG. 3, a photograph of the carotid bifurcation is shown. It was apparent that the diameter of the external carotid arterial sac was larger in FIG. 3(a-2) in which the vascular treatment material of Example 1 was placed. The orifice of the external carotid artery was that of the original external carotid artery, and an end of the vascular treatment material of Comparative Example 1 is exposed clearly to blood flow and the circumference of the vascular treatment material has transparency (FIG. 3(b-1)), whereas, an end of the vascular treatment material of Example 1 is covered by shiny tissue and a white tissue exists circumferentially (FIG. 3(b-2)), which can be apparently understood.

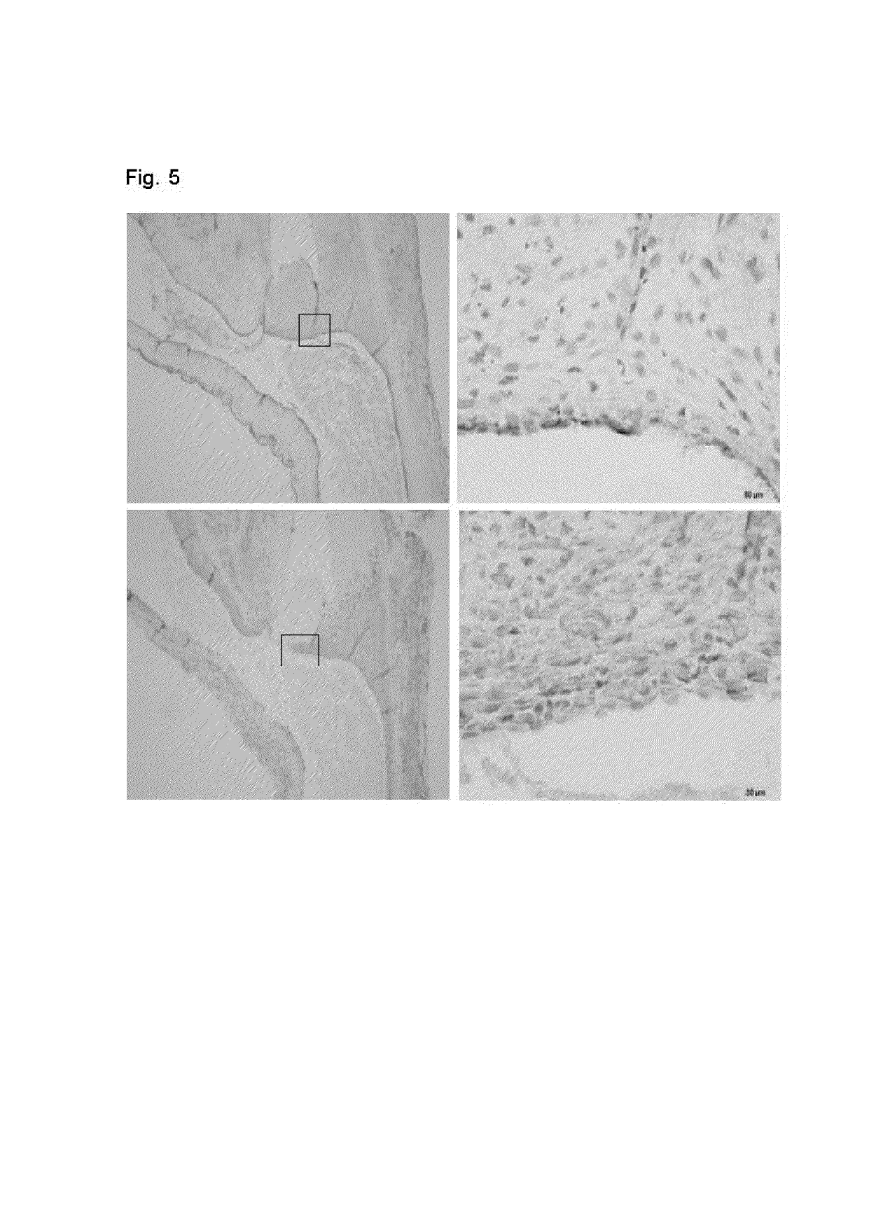

The taken out blood vessel was incised at 2 mm from the bifurcation and the cross section of the incised portion was stained with eosin hematoxylin (HE) and Masson trichrome (MT), and then the images are shown in FIGS. 4 to 6.

As is apparent from FIGS. 4 to 6, in the group using the vascular treatment material of Example 1, a cavity regarded as a residue of a blood flow in the aneurysm decreased and organization in the aneurysm made progress after 14 days. Sufficient organization was confirmed after 28 days. Neither vascular wall disorder nor occlusion of the internal carotid artery and the common carotid artery was recognized.

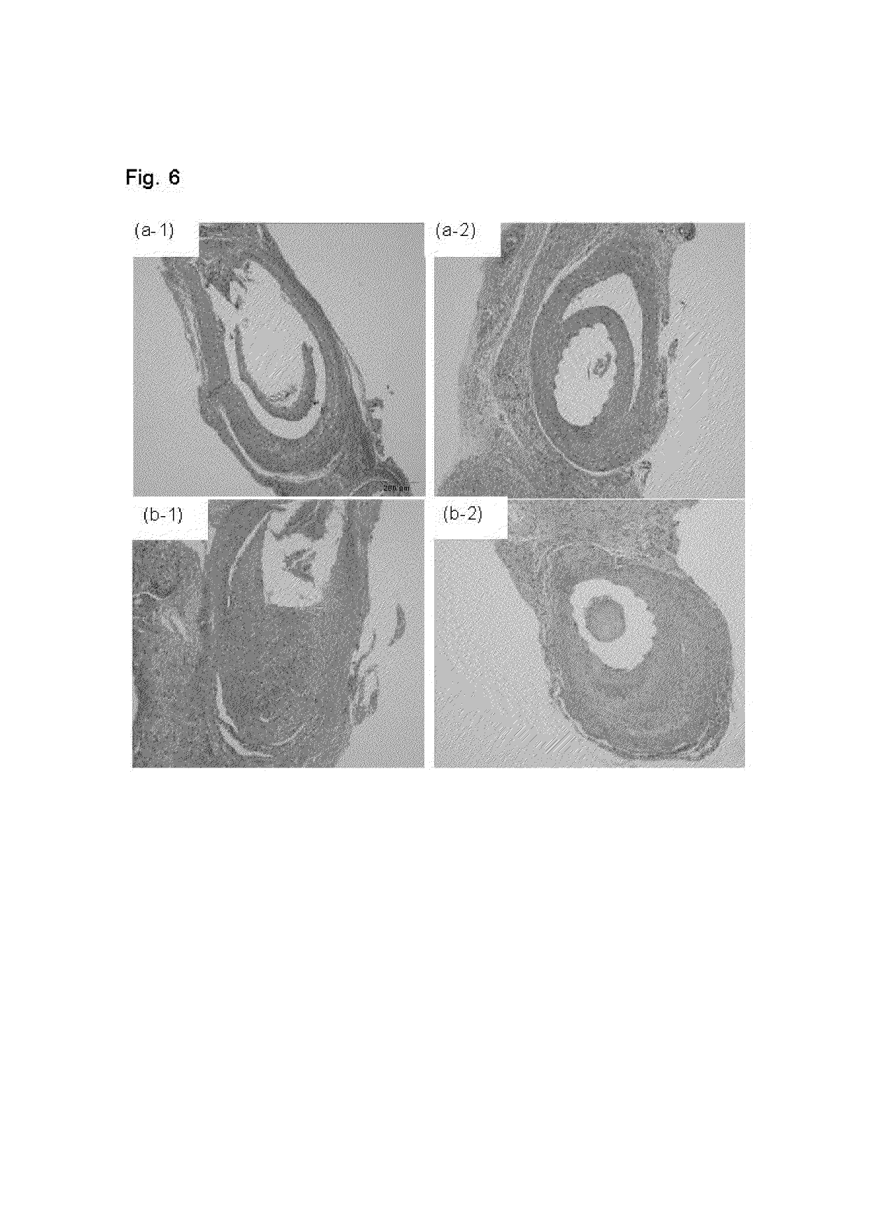

In contrast, in the group using the vascular treatment material of Comparative Example 1, a cavity regarded as a residue of a blood flow was recognized in the aneurysm even after 14 days. Such cavity was recognized particularly apparently in FIG. 6(a-1) and FIG. 6(a-2). While thin organization was recognized on the surface of the coil of the vascular treatment material of Comparative Example 1, only thickening of the endovascular film entirely occurred and an occlusion ratio was insufficient.

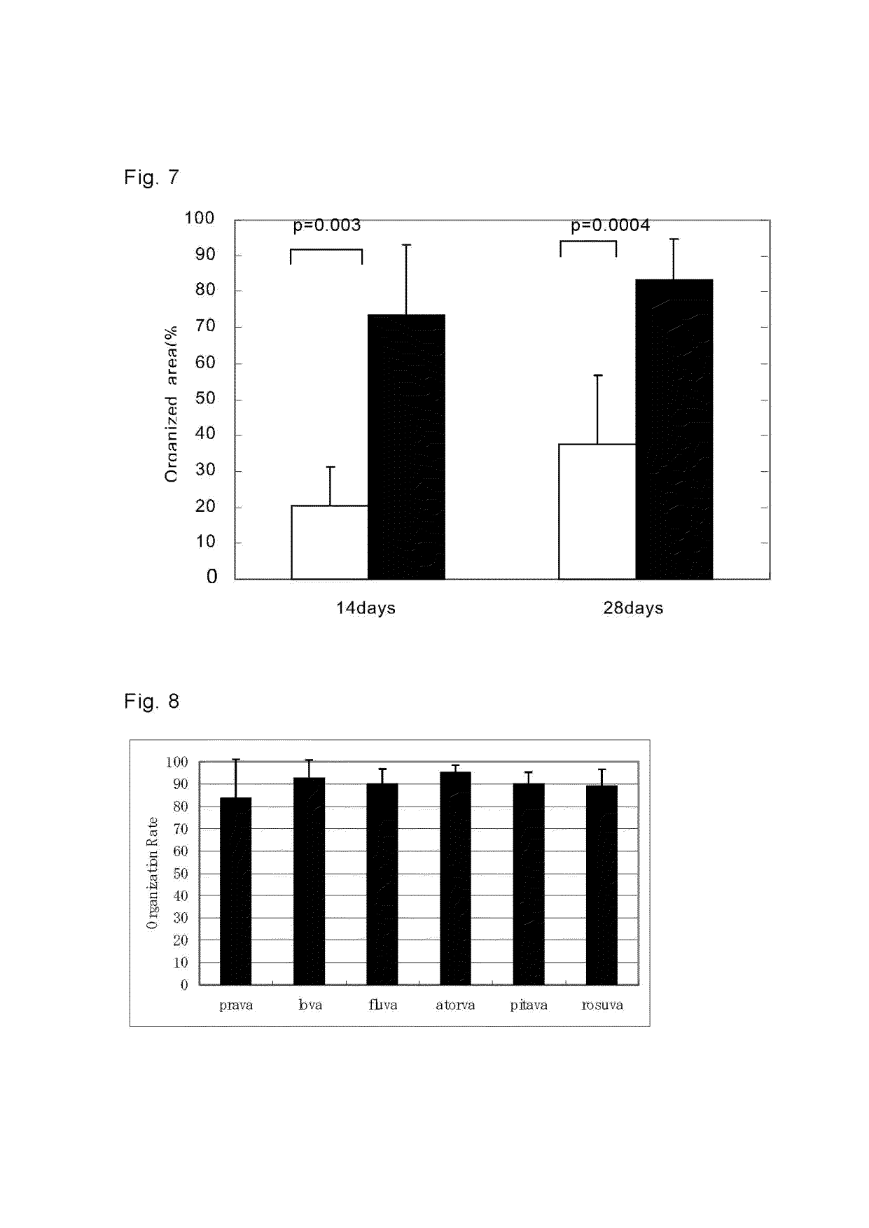

In FIGS. 4 to 6, the organized site seemed as if cell nucleus aggregated in the case of HE staining, and was stained blue in the case of MT staining. In contrast, the site where blood was coagulated seemed to be lack of cell nucleus in the case of HE staining, and was stained red or orange in the case of MT staining. Utilizing this, the rates occupied by the organized site were calculated by image analysis using a computer. The results are shown in FIG. 7.

As shown in FIG. 7, on the 14th day after placing the vascular treatment material of Example 1, the organization rate occupying the external carotid arterial sac was 73.6±19.4%. In contrast, on the 14th day after placing the vascular treatment material of Comparative Example 1, the organization rate occupying the external carotid arterial sac was 20.5±10.7%. Therefore, when the vascular treatment material of Example 1 was used, remarkably high organization rate was obtained. On the 28th day after placing the vascular treatment material of Example 1, the organization rate occupying the external carotid arterial sac was 83.4±11.1%. In contrast, on the 28th day after placing the vascular treatment material of Comparative Example 1, the organization rate occupying the external carotid arterial sac was 37.4±20.6%, and remarkably high organization rate was given when the vascular treatment material of Example 1 was used.

### Example 2

In the same manner as in Example 1, except that pravastatin was used in place of simvastatin, a vascular treatment material of Example 2 was obtained. Thereafter, organizability (or performance of organization) was evaluated using the external carotid artery of the Wister rats as the carotid bifurcation model. HE stained and MT stained images were evaluated by image analysis using a computer. The rate occupied by the organized site was significantly higher than that of Comparative Example 1. As shown in FIG. 8, on the 14th day after placing the vascular treatment material of Example 2, the organization rate occupying the external carotid arterial sac was 83.5±17.6%.

### Example 3

In the same manner as in Example 1, except that lovastatin was used in place of simvastatin, a vascular treatment material of Example 3 was obtained. Thereafter, organizability was evaluated using the external carotid artery of the Wister rats as the carotid bifurcation model. HE stained and MT stained images were evaluated by image analysis using a computer. The rate occupied by the organized site was significantly higher than that of Comparative Example 1. As shown in FIG. 8, on the 14th day after placing the vascular treatment material of Example 3, the organization rate occupying the external carotid arterial sac was 92.5±8.2%.

### Example 4

In the same manner as in Example 1, except that fluvastatin was used in place of simvastatin, a vascular treatment material of Example 4 was obtained. Thereafter, organizability was evaluated using the external carotid artery of the Wister rats as the carotid bifurcation model. HE stained and MT stained images were evaluated by image analysis using a computer. The rate occupied by the organized site was significantly higher than that of Comparative Example 1. As shown in FIG. 8, on the 14th day after placing the vascular treatment material of Example 4, the organization rate occupying the external carotid arterial sac was 90.0±6.7%.

### Example 5

In the same manner as in Example 1, except that atorvastatin was used in place of simvastatin, a vascular treatment material of Example 5 was obtained. Thereafter, organizability was evaluated using the external carotid artery of the Wister rats as the carotid bifurcation model. HE stained and MT stained images were evaluated by image analysis using a computer. The rate occupied by the organized site was significantly higher than that of Comparative Example 1. As shown in FIG. 8, on the 14th day after placing the vascular treatment material of Example 5, the organization rate occupying the external carotid, arterial sac was 95.4±3.5%.

### Example 6

In the same manner as in Example 1, except that pitavastatin was used in place of simvastatin, a vascular treatment material of Example 6 was obtained. Thereafter, organizability was evaluated using the external carotid artery of the Wister rats as the carotid bifurcation model. HE stained and MT stained images were evaluated by image analysis using a computer. The rate occupied by the organized site was significantly higher than that of Comparative Example 1. As shown in FIG. 8, on the 14th day after placing the vascular treatment material of Example 6, the organization rate occupying the external carotid arterial sac was 90.2±5.3%.

### Example 7

In the same manner as in Example 1, except that rosuvastatin was used in place of simvastatin, a vascular treatment material of Example 7 was obtained. Thereafter, organizability was evaluated using the external carotid artery of the Wister rats as the carotid bifurcation model. HE stained and MT stained images were evaluated by image analysis using a computer. The rate occupied by the organized site was significantly higher than that of Comparative Example 1. As shown in FIG. 8, on the 14th day after placing the vascular treatment material of Example 7, the organization rate occupying the external carotid arterial sac was 89.2±7.5%.

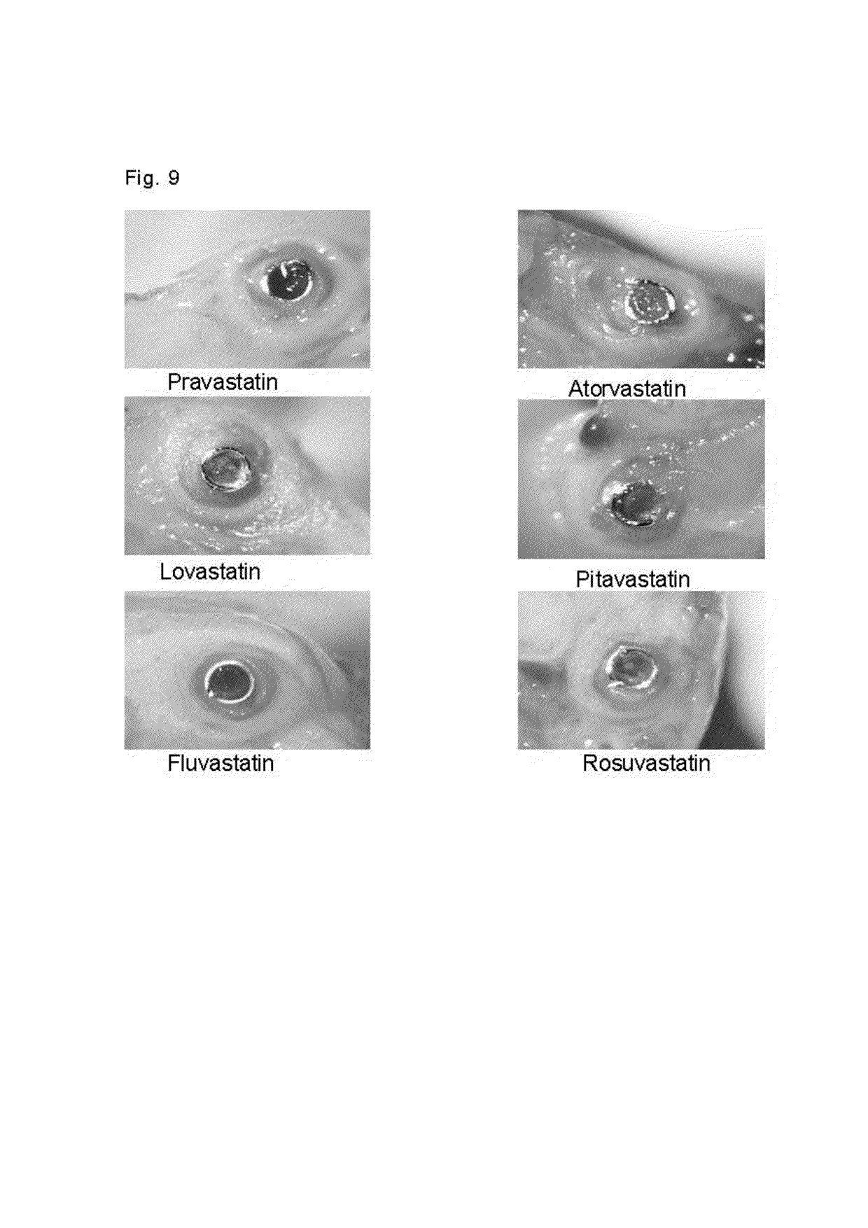

Furthermore, when the vascular treatment materials of Examples 2 to 7 were used, photographs of cross sections, each of which was obtained by cutting the aneurysm together with the coil at the position of 2 mm from the orifice of the aneurysm, are shown in FIG. 9. The photographs (upper left to lower left) showed the cross sections in case of using Examples 2 to 4, and the photographs (upper right to lower right) showed the cross sections in case of using Examples 5 to 7. Each of the photographs showed the cross sections on the 2nd week after implantation. When compared with the cross section of Comparative Example 1 shown in FIG. 3(b-1), it was apparent that the circumference of the coil was apparently surrounded by a white tissue and organization was significantly accelerated when the vascular treatment materials of Examples 2 to 7 were used.

### INDUSTRIAL APPLICABILITY

According to the present invention, it is possible to provide a vascular treatment material which is remarkably easily produced, and also can exert high organization acceleration effect.

### DESCRIPTION OF REFERENCE NUMERAL

1: Vascular treatment material (or material for curing blood vessel)
2: Blood vessel
3: Aneurysm
4: Fibroblasts, Smooth muscle cells
5: Vascular endothelial cells

1. A vascular treatment material comprising a coil on which statin is loaded.

2. The treatment material according to claim 1, wherein a wire forming the coil is made of at least one kind of metal selected from platinum, tungsten, gold, cobalt, chromium, titanium, niobium, aluminum, tantalum, iron and nickel.

3. The treatment material according to claim 1, wherein statin includes at least one kind selected from simvastatin, pravastatin, fluvastatin, lovastatin, mevastatin, cerivastatin, atorvastatin, pitavastatin and rosuvastatin.

4. The treatment material according to claim 1, wherein the treatment material is placed in a blood vessel for embolization treatment, or the treatment material accelerates organization for prevention of recanalization of aneurysm or for prevention of rupture of aneurysm.

5. A method for producing the treatment material according to claim 1, wherein the method comprises a step of immersing a coil in a statin-containing solution and then drying the coil, or coating a coil with a statin solution and then drying the coil.

6. The treatment material according to claim 2, wherein statin includes at least one kind selected from simvastatin, pravastatin, fluvastatin, lovastatin, mevastatin, cerivastatin, atorvastatin, pitavastatin and rosuvastatin.

7. The treatment material according to claim 2, wherein the treatment material is placed in a blood vessel for embolization treatment, or the treatment material accelerates organization for prevention of recanalization of aneurysm or for prevention of rupture of aneurysm.

8. A method for producing the treatment material according to claim 2, wherein the method comprises a step of immersing a coil in a statin-containing solution and then drying the coil, or coating a coil with a statin solution and then drying the coil.
